(19) United States
(12) Patent Application Publication
(10) Pub. No.: US 2007/0286766 A1
(43) Pub. Date: Dec. 13, 2007

(54) Apparatus for cleaning exhaust part and vacuum pump of reaction chamber for semiconductor device and LCD manufacturing equipment

(75) Inventor: Byoung-Chun Choi, Suwon-si (KR)

Correspondence Address: OLIFF & BERRIDGE, PLC, P.O. BOX 19928, ALEXANDRIA, VA, 22320, US

(73) Assignee: TERATECH CO., LTD., Gyeonggi-do (KR)

(21) Appl. No.: 11/594,065

(22) Filed: Nov. 8, 2006

(30) Foreign Application Priority Data
Jun. 12, 2006 (KR) 2006-52535

(51) Int. Cl. A61L 2/00

(52) U.S. Cl. 422/23

(57) ABSTRACT

Disclosed is an apparatus for cleaning an exhaust line of reaction chambers. The apparatus comprises plasma chambers into which a fluorine or chlorine gas is induced from a gas supply unit as a source of fluorine or chlorine radicals for removing solid deposits in the exhaust line, and a plasma source supply mechanism to convert the fluorine or chlorine gas into the fluorine or chlorine radicals through application of power to the fluorine or chlorine gas. The plasma source supply mechanism comprises an RF generator, antennas surrounding the plasma chamber to convert the fluorine or chlorine gas into the fluorine or chlorine radicals after receiving RF power, a relay switch for selection of RF supply to connect the RF generator with the plasma chambers, and a controller to control the RF power. The radicals are supplied to the exhaust line, and serve to remove the solid deposits.

### BACKGROUND OF THE INVENTION

1. Field of the Invention

The present invention relates to an apparatus for cleaning an exhaust part and a vacuum pump of a reaction chamber. More particularly, the present invention relates to an apparatus for cleaning an exhaust line including an exhaust part and a vacuum pump of a reaction chamber, which removes solid deposits formed in the reaction chamber during a deposition or etching process by use of fluorine or chlorine radicals.

2. Description of the Related Art

Generally, among semiconductor manufacturing processes, a cleaning process is one of important processes which fundamentally require stability of process. Recently, with rapid advance of semiconductor device manufacturing technology, it has been demanded to provide a technique capable of forming and processing ultra fine patterns. For this purpose, it is necessary to provide ultra clean environment, which in turn requires a surface treatment technique, a cleaning process, etc. to maintain the ultra clean environment. In this regard, it can be assured that a general substrate cleaning technique is already established, which can remove contaminants from a surface of a semiconductor substrate without contaminating the surface thereof. Furthermore, it is necessary to provide a cleaning technique for installations, such as a reaction chamber and subsidiary components thereof, used for manufacturing a semiconductor device through processing of a semiconductor substrate, such as deposition and etching. Since various processes are directly carried out on the substrate within the reaction chamber, cleaning of the reaction chamber is periodically performed to prevent contamination of the semiconductor substrate, and a technique for this process is also established in the art. However, there have been few suggestions of a cleaning technique for the subsidiary components of the reaction chamber, such as an exhaust part, a vacuum pump, etc. due to recognition that such subsidiary components are not directly related to the contamination on the substrate.

FIG. 1 is a schematic view of an exhaust part of a conventional reaction chamber.

Referring to FIG. 1, an exhaust pipe 110 is connected to an exhaust port of a reaction chamber 100 where deposition or etching of an oxide layer, nitride layer, polysilicon layer, metal layer, etc. is performed on a semiconductor substrate. The exhaust pipe 110 is provided at the middle thereof with first and second gate valves 120 and 122, and a pressure adjustment valve 124 to control flow and pressure of exhaust gas, and with a turbo molecular pump 123 between the first gate valves 120 and the pressure adjustment valve 124. A vacuum pump 130 is positioned at the next stage of the second gate valve 122 to evacuate the reaction chamber 100 and discharge the exhaust gas to an outside. The exhaust gas passes through the vacuum pump 130, and is discharged to atmosphere after being subjected to treatment by means of a scrubber 140 so as to satisfy standards against hazardous material.

Solid deposits, by-products of the deposition or etching process in the reaction chamber 100 of FIG. 1, are accumulated in the exhaust pipe 110, causing a hindrance to the pressure adjustment valve 124 acting to adjust the pressure of the reaction chamber 100. Otherwise, the solid deposits are accumulated in the turbo molecular pump 123, causing a reduction in capability or inoperability thereof. Therefore, semiconductor manufacturing equipment suffers from reduction in operational rate. In addition, in the event of cleaning the subsidiary components by an operator, since it is necessary to use chemical agents, there are problems of inconvenience and danger in operation.

It can be suggested to clean the subsidiary components in such a way of directly generating radicals for cleaning the exhaust pipe 100 within the reaction chamber 100 as shown in FIG. 1, followed by discharging the radicals through the exhaust pipe 100. In this case, however, the radicals are likely to be recombined via pressure difference between the reaction chamber 100 and the exhaust pipe 100, making it substantially impossible to clean the exhaust pipe 100.

### SUMMARY OF THE INVENTION

The present invention has been made to solve the above problems, and it is an object of the present invention to provide a cleaning apparatus which can remove solid deposits accumulated in an exhaust part of a reaction chamber without manual operation.

It is another object of the present invention to provide the apparatus for the exhaust part and vacuum pump of the reaction chamber, which allows a clean state and constant pressure of the vacuum exhaust part to be maintained for the purpose of an accurate process control, thereby enabling improvement in yield of semiconductor manufacturing equipment, minimizing reduction in operable rate thereof, and reducing maintenance costs for cleaning operation.

In accordance with one aspect of the present invention, the above and other objects can be accomplished by the provision of an apparatus for cleaning exhaust parts and vacuum pumps of plural reaction chambers, comprising: a plurality of plasma chambers into which a fluorine or chlorine gas is induced from a gas supply unit as a source of fluorine or chlorine radicals used for removing solid deposits accumulated in the exhaust part and vacuum pump by reaction gas for a process performed in each of the reaction chambers; and a plasma source supply mechanism to convert the fluorine or chlorine gas into the fluorine or chlorine radicals through application of plasma power to the fluorine or chlorine gas induced into each of the plasma chambers, the plasma source supply mechanism comprising an RF generator, a plurality of antennas, each surrounding a corresponding plasma chamber to convert the fluorine or chlorine gas into the fluorine or chlorine radicals within each of the plasma chambers after receiving RF power supplied from the RF generator, a relay switch for selection of RF supply to connect the RF generator with each of the plasma chambers, and a controller to control the RF power generated by the RF generator, wherein the radicals converted by the plasma source supply mechanism are supplied to each of the exhaust pipes, and serve to remove the solid deposits accumulated therein.

The gas supply unit may be provided with a flow restrictor. The gas supply unit may be provided with a pressure display and an automatic cut-off switch to detect leakage of the supply gas and automatically stop supply of the gas if the leakage of the supply gas is detected.

The apparatus may further comprise an RF matching box between each of the antennas and the relay switch for selection of RF supply. The RF matching box may comprise a variable capacitor.

The exhaust parts may comprise at least one selected from a vacuum line, an exhaust pipe, a pressure control valve, a turbo molecular pump, a gate valve and a vacuum pump.

The RF generator may generate RF power of 0.2˜2 kW with a frequency of 13.56 MHz.

### BRIEF DESCRIPTION OF THE DRAWINGS

The foregoing and other objects and features of the present invention will be more clearly understood from the following detailed description taken in conjunction with the accompanying drawings, in which:

FIG. 1 is a schematic view of an exhaust part of a conventional reaction chamber;

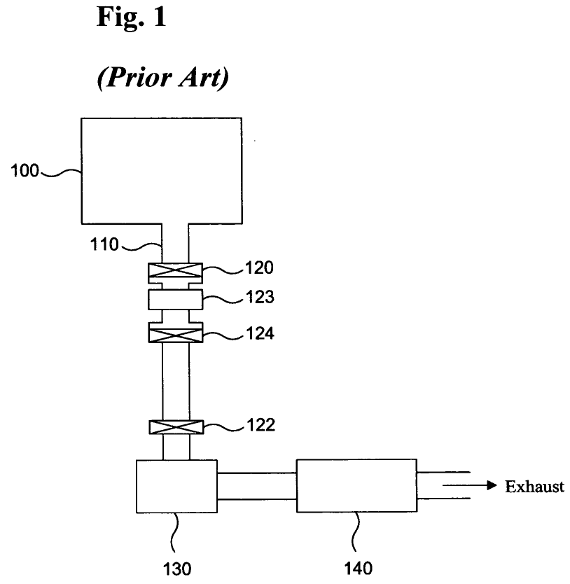

FIG. 2 is a schematic view of an apparatus for cleaning an exhaust part and a vacuum pump of a reaction chamber according to one embodiment of the present invention; and

FIG. 3 is a cross-sectional view of an inner construction of a fluorine or chlorine radical generator of the apparatus shown in FIG. 2.

### DETAILED DESCRIPTION OF THE PREFERRED EMBODIMENTS

Preferred embodiments of the present invention will be described in detail with reference to the accompanying drawings, in which like components are denoted by the same reference numerals, and repetitious descriptions thereof will be omitted.

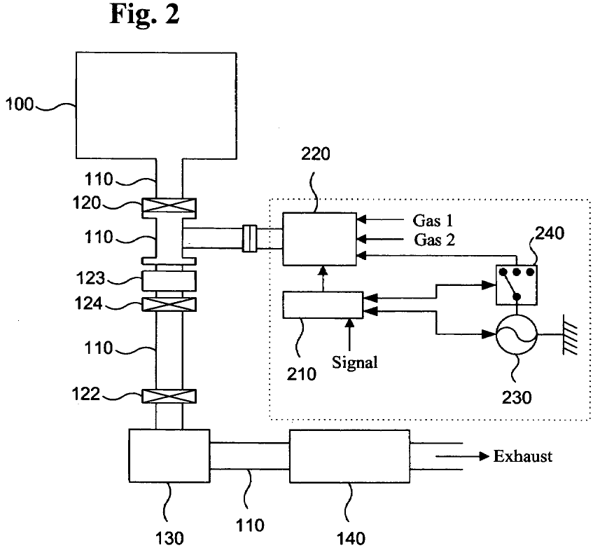

FIG. 2 is a schematic view of an apparatus for cleaning an exhaust part and a vacuum pump of a reaction chamber according to one embodiment of the present invention.

Referring to FIG. 2, the apparatus 200 (which will hereinafter be referred to as a “cleaning apparatus”) for cleaning the exhaust part and vacuum pump of the reaction chamber according to the present invention is installed to an exhaust pipe 110 between a first gate valve 120 and a pressure control valve 124 by means of flange coupling. However, it should be noted that an installation location of the cleaning apparatus 200 is not limited to the above location. Thus, the cleaning apparatus 200 can be coupled to any portion of the exhaust pipe 110. The cleaning apparatus 200 comprises a fluorine or chlorine radical generator 220 into which both Gas 1 and Gas 2 are supplied. Gas 1 may be selected among fluorine contained gases, for example, NF₃ or PFC such as C₃F₈, C₄F₈, SF₆, etc. In addition, Gas 1 may be selected among chlorine contained gases, for example, Cl₂, HCl, BCl₃, CCl₄, etc. Gas 2 may be selected among O₂ and Ar. The fluorine or chlorine gas and O₂ or Ar supplied into the cleaning apparatus 200 are converted into fluorine or chlorine radicals by means of RF power of 0.2˜2 kW at a frequency of 13.56 MHz supplied from an RF generator 230 via a relay switch 240 for selection of RF supply by a controller 210. In other words, the RF generator 230 acts as an inductively coupled plasma source supply mechanism which induces plasma by use of the fluorine or chlorine gas and O₂ or Ar. The controller 210 allows the RF power to be selectively supplied to a target radical supply unit through control of the RF power generated by the RF generator 230 and a switching operation of the relay switch 240 in response to signals from an exterior. Furthermore, the controller 210 serves to control supplying and blocking of Gases 1 and 2. After being produced by this mechanism, the fluorine or chlorine radicals etch and remove solid deposits accumulated in the pressure control valve 124, turbo molecular pump 123, second gate valve 122, vacuum pump 130, scrubber 140, and the exhaust pipe 110 disposed therebetween during a deposition or etching process within a reaction chamber 100 while sequentially passing therethrough.

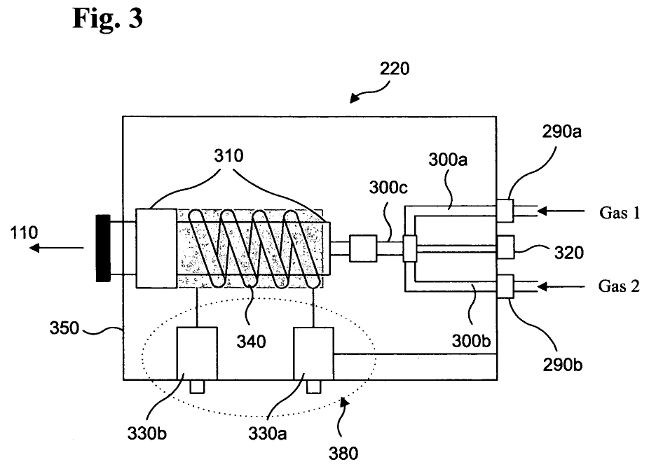

FIG. 3 is a cross-sectional view of an inner construction of the fluorine or chlorine radical generator 220 of the apparatus shown in FIG. 2.

Referring to FIG. 3, major components of the fluorine or chlorine radical generator 220 are contained in a case 350. In a state of being restricted in maximum passage flux by means of flow restrictors 290a and 290b, Gas 1, that is, the fluorine or chlorine gas, and Gas 2, that is, O₂ or Ar, pass through the first and second gas supply pipes 300a and 300b, respectively. Then, the fluorine or chlorine gas and O₂ or Ar are mixed in a single gas supply pipe 300c, and flow into a plasma chamber 310. The plasma chamber 310 has an antenna 340 positioned on an outer wall to helically surround the plasma chamber 310. The antenna 340 is supplied with RF power from the RF generator 230 through an RF matching box 380 via the relay switch 240 for selection of RF supply, and serves to produce fluorine or chlorine radicals by use of the fluorine or chlorine gas and O₂ or Ar within the plasma chamber 310. In this embodiment, the RF matching box 380 comprises two variable capacitors 330a and 330b. After being produced by these components, the fluorine or chlorine radicals leave are discharged from the plasma chamber 310, and then enter the exhaust pipe 110, finally etching and removing solid deposits accumulated in the pressure control valve 124, turbo molecular pump 123, second gate valve 122, vacuum pump 130, scrubber 140, and the exhaust pipe 110 disposed therebetween while sequentially passing therethrough.

Meanwhile, the fluorine or chlorine radical generator 220 is provided with a pressure display and an automatic cut-off switch 320 to detect leakage of the gas from the first and second gas supply pipes 300a and 300b, and then automatically stop supply of the gas if the leakage of the supply gas is detected.

In the above description, the cleaning apparatus according to the present invention is described as being installed in a single reaction chamber. However, since it is normal to perform various processes in plural reaction chambers, it should be noted that the present invention is not limited to the above arrangement. In this regard, even for the purpose of removing solid deposits accumulated in an exhaust part and a vacuum pump provided to each of the plural reaction chambers during semiconductor device manufacturing processes, it is not necessary to provide a plurality of components for all the cleaning apparatuses installed to the reaction chambers. In other words, although the plasma chambers 310 and antennas 340 are installed to associated reaction chambers, respectively, the cleaning apparatus may have a single RF generator 230, a single relay switch 240 for selection of RF supply, and a single controller 210 such that RF power can be applied to an exhaust pipe of a target reaction chamber among the plural reaction chambers pursuant to a state of the RF switch 240.

As such, the cleaning apparatus having the construction as described above according to the embodiment of the present invention is preferably controlled to be used only in a period while a main process is not performed in the reaction chamber, thereby improving an operational rate of semiconductor manufacturing equipment. Needless to say, the cleaning apparatus can be operated during the main process.

As described above, in manufacturing of the semiconductor devices, the conventional equipment has a problem in that, with an increase in size of a semiconductor substrate, a great amount of gas is used in the reaction chamber used for the manufacturing process, causing accumulation of by-products in the exhaust part and vacuum pump of the reaction chamber.

According to the present invention, however, the cleaning apparatus increases an operational rate of equipment, thereby enhancing productivity, compared with the conventional technique which requires an operator to stop the equipment and manually remove the solid deposits accumulated in the exhaust part and vacuum pump of the reaction chamber. Furthermore, the cleaning apparatus enables a dry-etching gas for removing the solid deposits to be supplied via a separate line other than the reaction chamber, and thus eliminates requirement to change process conditions of the reaction chamber, thereby improving reliability in process of the equipment, while reducing maintenance costs due to malfunction of devices and components, such as an exhaust pipe, of the reaction chamber.

It should be understood that the embodiments and the accompanying drawings have been described for illustrative purposes and the present invention is limited only by the following claims. Further, those skilled in the art will appreciate that various modifications, additions and substitutions are allowed without departing from the scope and spirit of the invention as set forth in the accompanying claims.

What is claimed is:

1. An apparatus for cleaning exhaust parts and vacuum pumps of plural reaction chambers, comprising:
a plurality of plasma chambers into which a fluorine or chlorine gas is induced from a gas supply unit as a source of fluorine or chlorine radicals used for removing solid deposits accumulated in the exhaust part and vacuum pump by reaction gas for a process performed in each of the reaction chambers; and
a plasma source supply mechanism to convert the fluorine or chlorine gas into the fluorine or chlorine radicals through application of plasma power to the fluorine or chlorine gas induced into each of the plasma chambers, the plasma source supply mechanism comprising an RF generator, a plurality of antennas, each surrounding a corresponding plasma chamber to convert the fluorine or chlorine gas into the fluorine or chlorine radicals within each of the plasma chambers after receiving RF power supplied from the RF generator, a relay switch for selection of RF supply to connect the RF generator with each of the plasma chambers, and a controller to control the RF power generated by the RF generator,
wherein the radicals converted by the plasma source supply mechanism are supplied to each of the exhaust pipes, and serve to remove the solid deposits accumulated therein.

2. The apparatus according to claim 1, wherein the gas supply unit is provided with a flow restrictor.

3. The apparatus according to claim 1, wherein the gas supply unit is provided with a pressure display and an automatic cut-off switch to detect leakage of the supply gas and automatically stop supply of the gas if the leakage of the supply gas is detected.

4. The apparatus according to claim 1, further comprising:
an RF matching box between each of the antennas and the relay switch for selection of RF supply.

5. The apparatus according to claim 4, wherein the RF matching box comprises a variable capacitor.

6. The apparatus according to claim 1, wherein the RF generator generates RF power of 0.2˜2 kW with a frequency of 13.56 MHz.

7. The apparatus according to claim 1, wherein the exhaust parts comprises at least one selected from a vacuum line, an exhaust pipe, a pressure control valve, a turbo molecular pump, a gate valve and a vacuum pump.
